(19) United States
(12) Patent Application Publication
(10) Pub. No.: US 2017/0246092 A1
(43) Pub. Date: Aug. 31, 2017

(54) Zinc-Containing Compositions with Essential Oils

(71) Applicant: Colgate-Palmolive Company, New York, NY (US)

(72) Inventors: Michael PRENCIPE, West Windsor, NJ (US); Steven FISHER, Middlesex, NJ (US); Gary TAMBS, Belle Mead, NJ (US)

(73) Assignee: Colgate-Palmolive Company, New York, NY (US)

(21) Appl. No.: 15/507,330

(22) PCT Filed: Sep. 1, 2014
(86) PCT No.: PCT/US2014/053629

§ 371 Date: Feb. 28, 2017

(51) Int. Cl. A61K 8/27; A61K 8/33; A61Q 11/00; A61K 8/35; A61K 8/97; A61K 8/31; A61K 8/34

(52) U.S. Cl. CPC A61K 8/27; A61K 8/31; A61K 8/33; A61K 8/34; A61K 8/347; A61K 8/35; A61K 8/97; A61Q 11/00

(57) ABSTRACT

The present invention provides an oral care composition comprising: (a) at least one zinc ion source; and (b) at least two active ingredients, wherein said active ingredients are selected from one or more of the following groups: (i) monoterpenoid phenols; (ii) monoterpenoid aldehydes; (iii) sesquiterpenoid alcohols; (iv) active compounds from an extract of oregano; and (v) active compounds from an extract of rosemary.

### BACKGROUND

Dental plaque is a biofilm that adheres to tooth and other oral surfaces, particularly at the gingival margin, and is implicated in the occurrence of gingivitis, periodontitis, caries and other forms of periodontal disease. Various antibacterial agents can retard the growth of bacteria and thus reduce the formation of biofilm on oral surfaces. Zinc and other metal compounds/salts have been previously used as antibacterial agents. Without being bound by any theory, free zinc ions are believed to provide antibacterial efficacy by inhibition of glucose metabolism and/or interaction with the bacterial cell wall, reducing bacterial colonization of the oral cavity (as discussed in Cummins D., J Clin Periodontol 1991; 18; 455.-461). An insoluble zinc compound, zinc oxide, could also deliver strong antibacterial efficacy during tooth brushing.

It would be desirable to provide an oral care composition which exhibits even greater biofilm reduction efficacy than previously-known compositions.

### BRIEF SUMMARY

In a first aspect, the present invention provides an oral care composition comprising:

(a) at least one zinc ion source; and

(b) at least two active ingredients, wherein said active ingredients are selected from one or more of the following groups:
(i) monoterpenoid phenols,
(ii) monoterpenoid aldehydes,
(iii) sesquiterpenoid alcohols,
(iv) active compounds from an extract of oregano, and
(v) active compounds from an extract of rosemary.

Optionally, the total concentration of the at least one zinc ion source is from 0.1 to 4.0 weight %, based on the total weight of the composition. Further optionally, the total concentration of the at least one zinc ion source is from 0.5 to 2.0 weight %, based on the total weight of the composition.

Optionally, the total concentration of the at least two active ingredients is from 500 to 10,000 ppm. Further optionally, the total concentration of the at least two active ingredients is from 1000 to 5000 ppm.

Optionally, the monoterpenoid phenols are carvacrol and thymol. Optionally, the composition comprises carvacrol. Optionally, the composition comprises thymol.

Optionally, the monoterpenoid aldehyde is citral. Optionally, the composition comprises citral.

Optionally, the sesquiterpenoid alcohol is bisabolol. Optionally, the composition comprises bisabolol.

Optionally, the composition comprises bisabolol and c-itral. Further optionally, the ratio of citral to bisabolol is from 1:1 to 3:1 by weight; optionally about 2:1 by weight.

Optionally, the composition comprises bisabolol and carvacrol. Further optionally, the ratio of bisabolol to carvacrol is from 1:2 to 2:1 by weight; optionally about 1.5:1 by weight.

Optionally, the composition comprises bisabolol and thymol.

Optionally, the composition comprises citral and carvacrol.

Optionally, the composition comprises citral and thymol.

Optionally, the composition comprises carvacrol and thymol. Further optionally, the ratio of thymol to carvacrol is from 8:1 to 13:1 by weight; optionally about 11.25:1 by weight.

Optionally, the active compounds from an extract of oregano are carvacrol, thymol, limonene, pinene, p-cymene and caryophyllene. Further optionally, the oral care composition comprises an extract of oregano, the extract of oregano comprising carvacrol, thymol, limonene, pinene, p-cymene and caryophyllene.

Optionally, the oral care composition comprises an extract of oregano, the extract of oregano comprising 50 to 70 weight % carvacrol, 10 to 35 weight % thymol and 10 to 20 weight % p-cymene, based on the weight of the extract of oregano.

Optionally, the composition comprises bisabolol and the extract of oregano. Further optionally, the ratio of the extract of oregano to bisabolol is from 2:1 to 6:1 by weight; optionally about 4,4:1 by weight.

Optionally, the composition comprises citral and the extract of oregano.

Optionally, the active compounds from an extract of rosemary are α-pinene, β-pinene, borneol, bornyl acetate, camphor, camphene, 1,8-cineole and limonene. Further optionally, the oral care composition comprises an extract of rosemary, the extract of rosemary comprising α-pinene, β-pinene, borneol, bornyl acetate, camphor, camphene, 1,8-cineole and limonene.

Optionally, the oral care composition comprises an extract of rosemary, the extract of rosemary comprising 25 to 40 weight % α-pinene, 5 to 20 weight % β-pinene, and 10 to 25 weight % camphor, based on the weight of the extract of rosemary.

Optionally, the composition comprises bisabolol and the extract of rosemary. Further optionally, the ratio of the extract of rosemary to bisabolol is from 40:1 to 60:1 by weight; optionally about 48:1 by weight.

Optionally, the composition comprises citral and the extract of rosemary.

Optionally, the composition comprises carvacrol and the extract of rosemary. Further optionally, the ratio of the extract of rosemary to carvacrol is from 70:1 to 80:1 by weight; optionally about 75:1 by weight.

Optionally, the composition comprises thymol and the extract of rosemary.

Optionally, the composition comprises the extract of rosemary and an extract of oregano. Further optionally, the extract of oregano comprises carvacrol, thymol, limonene, pinene, p-cymene and caryophyllene.

Optionally, the extract of oregano comprises 50 to 70 weight % carvacrol, 10 to 35 weight % thyrnol and 10 to 20 weight % p-cymene, based on the weight of the extract of oregano.

Optionally, the at least one zinc ion source is selected from zinc citrate, zinc oxide, zinc acetate, zinc gluconate, zinc glycinate, zinc sulfate, zinc phosphate and sodium zinc citrate. Further optionally, the composition comprises zinc oxide and zinc citrate. Still further optionally, the molar ratio of zinc oxide to zinc citrate is from 0:5 to 3:1. Yet further optionally, the molar ratio of zinc oxide to zinc citrate is from 1:1 to 3:1, optionally about 2:1.

Optionally, the composition comprises from 0.1 to 2.0 weight % zinc oxide and from 0.20 to 1.0 weight % zinc citrate; optionally from 0.5 to 1.5 weight % zinc oxide and from 0.25 to 0.75 weight % zinc citrate.

Optionally, the composition is a dentifrice, a toothpaste, a gel, a tooth powder, a mouthwash, a mouthrinse, a lozenge, a tablet, a spray, a gum, or a film.

In a second aspect, the present invention provides an oral care composition of the present invention, for use in reducing or inhibiting biofilm formation in an oral cavity.

In a third aspect, the present invention provides a method of reducing or inhibiting biofilm formation in an oral cavity, the method comprising contacting the oral cavity with an oral care composition of the present invention.

In a fourth aspect, the present invention provides the use, in an oral care composition, of a combination of (a) at least one zinc ion source; and (b) at least two active ingredients, wherein said active ingredients are selected from one or more of the following groups: (i) monoterpenoid phenols; (ii) monoterpenoid aldehydes; (iii) sesquiterpenoid alcohols; (iv) active compounds from an extract of oregano; and (v) active compounds from an extract of rosemary; to reduce or inhibit biofilm formation in an oral cavity.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material. As referred to herein, “ppm” (parts per million) refers to ppm by weight unless otherwise indicated. In addition, all ratios expressed herein refer to ratios by weight unless otherwise indicated.

Unless otherwise specified, all experiments described herein are conducted at 25° C. and under atmospheric pressure.

As discussed above, it would be desirable to provide an oral care composition which exhibits greater biofilm reduction efficacy than previous oral care compositions.

Therefore, in one aspect of the present invention, there is provided an oral care composition comprising:

(a) at least one zinc ion source; and

(b) at least two active ingredients, wherein said active ingredients are selected from one or more of the following groups:
(i) monoterpenoid phenols,
(ii) monoterpenoid aldehydes,
(iii) sesquiterpenoid alcohols,
(iv) active compounds from an extract of oregano, and
(v) active compounds from an extract of rosemary.

In some embodiments, the total concentration of the at least two active ingredients in the oral care composition is from 50 to 12,000 ppm (0.005 to 1.2 weight %), from 100 to 12,000 ppm (0.01 to 1.2 weight %), from 500 to 10,000 ppm (0.05 to 1 weight %), from 700 to 6000 ppm (0.07 to 0.6 weight %), from 900 to 5500 ppm (0.09 to 0.55 weight %), from 1000 to 5000 ppm (0.1 to 0.5 weight %), from 3000 to 5000 ppm (0.3 to 0.5 weight %); or about 1000 ppm (0.1 weight %), about 3000 ppm (0.3 weight %), or about 5000 ppm (0.5 weight %). In some embodiments, the total concentration of the at least two active ingredients in the oral care composition is from 2500 to 5500 ppm (0.25 to 0.55 weight %), from 4500 to 5500 ppm (0.45 to 0.55 weight %), from 4800 to 5200 ppm (0.48 to 0.52 weight %), or about 5000 ppm (0.5 weight %).

Terpenes are hydrocarbons formally derived from the combination of one or more five-carbon isoprene units, CH₂═C(CH₃)CH═CH₂ (2-methyibuta-1,3-diene, molecular formula C₅H₈). The isoprene units may he linked together to form linear chains, or may be arranged to form rings (for example, benzene rings). Terpenoids (sometimes referred to as “isoprenoids”) can be thought of as being modified terpenes, e.g. containing additional functional groups (such as, for example, alcohol or aldehyde groups) or wherein methyl groups have been moved or removed. As for terpenes, terpenoids can be classified according to the number of isoprene units from which they are formally derived:

Hemiterpenoids: 1 isoprene unit (i.e. C₅ skeleton)

Monoterpenoids: 2 isoprene units (i.e. C₁₀ skeleton)

Sesquiterpenoids: 3 isoprene units (i.e. C₁₅ skeleton)

Diterpenoids: 4 isoprene units (i.e. C₂₀ skeleton)

Sesterterpenoids: 5 isoprene units (i.e. C₂₅ skeleton)

Triterpenoids: 6 isoprene units (i.e. C₃₀ skeleton)

Tetraterpenoids: 8 isoprene units (i.e. C₄₀ skeleton)

A monoterpenoid aldehyde which may be used in the compositions of the present invention is citral (also known as 3,7-dimethyl-2,6-octadienal or lemonal). There are two double-bond stereoisomers of citral: the E-isomer (which is known as geranial or citral A); and the Z-isomer (which is known as neral or citral B). In the present invention, the citral is a mixture of these two isomers at a ratio of 60:40 E-isomer to Z-isomer. The structures of these two isomers is shown below:

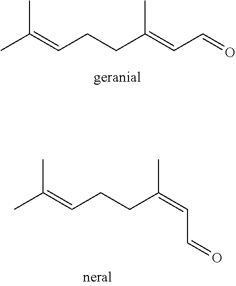

In some embodiments, the oral care composition comprises citral. In some embodiments, citral is present in the oral care composition in an amount of from 500 to 4000 ppm, 600 to 3600 ppm, 1500 to 3500 ppm, 2000 to 3500 ppm, or 3200 to 3400 ppm.

Monoterpenoid phenols which may be used in the compositions of the present invention include carvacrol and thymol. Thymol (also known as 2-isopropyl-5-methylphenol) is isomeric with carvacrol (also known as 5-isopropyl-2-methylphenol, or cymophenol). The structures of these two compounds are shown below:

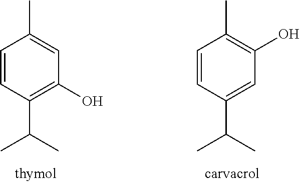

In some embodiments, the oral care composition comprises carvacrol. In other embodiments, the oral care composition comprises thymol.

In some embodiments, carvacrol is present in the oral care composition in an amount of from 5 to 3000 ppm, from 10 to 2500 ppm, or from 10 to 2000 ppm. In some embodiments, carvacrol is present in the oral care composition in an amount of from 80 to 2000 ppm. In other embodiments, carvacrol is present in the oral care composition in an amount of from 10 to 75 ppm, from 35 to 70 ppm, or from 50 to 70 ppm.

In some embodiments, thymol is present in the oral care composition in an amount of from 500 to 5000 ppm, from 800 to 4800ppm, from 900 to 4600 ppm, from 2600 to 4600 ppm, or from 4500 to 4600 ppm.

In certain embodiments, the oral care composition comprises thymol and carvacrol. In some embodiments, the ratio of thymol to carvacrol is from 5:1 to 15:1 by weight, from 6:1 to 14:1 by weight, from 8:1 to 13:1 by weight, from 9:1 to 12.5:1 by weight, from 10:1 to 12:1 by weight, or about 11.25:1 by weight. In some embodiments, the oral care composition comprises from 50 to 450 ppm carvacrol and from 800 to 4800 ppm thymol; from 80 to 410 ppm carvacrol and from 900 to 4600 ppm thymol; from 200 to 410 ppm carvacrol and from 2600 to 4600 ppm thymol; or from 390 to 410 ppm carvacrol and from 4500 to 4600 ppm thymol.

A sesquiterpenoid alcohol which may be used in the compositions of the present invention is bisabolol (also known as levomenol). There are two structural isomers of bisabolol: α-bisabolol and β-bisabolol. The structures of these isomers are shown below:

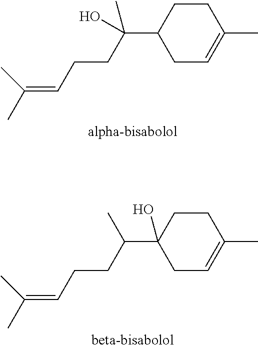

In certain embodiments of the present invention, the isomer of bisabolol which is present in the oral care compositions is α-bisabolol. There are two enantiomers of α-bisabolol: α-(−)-bisabolol and α-(+)-bisabolol. Of these enantiomers, α-(−)-bisabolol (illustrated below) is naturally occurring, and is found in German chamomile (Matricaria recutita) and Myoporum crassifolium. α-(+)-Bisabolol is also found in nature, but is rare.

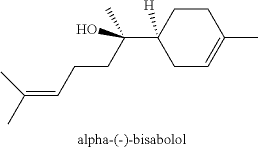 [SMILES: CC(C)=CCC[C@@](C)(O)[C@@H]1CC=C(C)CC1]

Synthetic bisabolol is usually a racemic mixture of the two enantiomers i.e. α-(±)-bisabolol. In certain embodiments of the present invention, the bisabolol which is present in the oral care compositions is racemic α-(±)-bisabolol.

In some embodiments, the oral care composition comprises bisabolol. In some embodiments, bisabolol is present in the oral care composition in an amount of from 10 to 3500 ppm, from 15 to 3100 ppm, or from 20 to 3050 ppm. In certain embodiments, bisabolol is present in the oral care composition in an amount of from 10 to 1000 ppm, from 20 to 950 ppm, or from 100 to 950 ppm. In other embodiments, bisabolol is present in the oral care composition in an amount of from 300 to 3500 ppm, from 1000 to 3200 ppm, or from 1500 to 3100 ppm.

In some embodiments, the oral care composition comprises bisabolol and citral. In certain embodiments, the ratio of citral to bisabolol is from 0.5:1 to 4:1 by weight, from 1:1 to 3:1 by weight, from 1.5:1 to 2.5:1 by weight, from 1.75:1 to 2.25:1 by weight, or about 2:1 by weight. In some embodiments, the oral care composition comprises from 300 to 2000 ppm bisabolol and from 500 to 3500 ppm citral; from 300 to 1700 ppm bisabolol and from 600 to 3400 ppm citral; from 1000 to 1700 ppm bisabolol and from 1800 to 3400 ppm citral; or from 1500 to 1700 ppm bisabolol and from 3100 to 3400 ppm citral.

In some embodiments, the oral care composition comprises bisabolol and carvacrol. In certain embodiments, the ratio of bisabolol to carvacrol is from 1:3 to 3:1 by weight, from 0.5:1 to 2:1 by weight, from 1:1 to 2:1 by weight, from 1.25:1 to 1.75:1 by weight, or about 1.5:1 by weight. In some embodiments, the oral care composition comprises from 500 to 3500 ppm bisabolol and from 250 to 2500 ppm carvacrol; from 600 to 3100 ppm bisabolol and from 300 to 2000 ppm carvacrol; from 1500 to 3100 ppm bisabolol and from 1000 to 2000 ppm carvacrol; or from 3000 to 3100 ppm bisabolol and from 1900 to 2000 ppm carvacrol,

In some embodiments, the oral care composition comprises bisabolol and thymol.

In some embodiments, the composition comprises citral and carvacrol.

In some embodiments, the composition comprises citral and thymol.

In some embodiments, the active compounds from an extract of oregano are cyclic monoterpenes, bicyclic monoterpenes, monoterpenoid phenols, bicylic sesquiterpenoids, and alkylbenzene compounds. In some embodiments, the active compounds from an extract of oregano are carvacrol, thymol, limonene, pinene, p-cymene and caryophyllene. Limonene is a cyclic monoterpene, pinene is a bicyclic monoterpene, caryophyllene is a bicylic sesquiterpenoid, and p-cymene is an alkylbenzene compound related to cyclic monoterpenes. In some embodiments, the oral care composition comprises an extract of oregano, the extract of oregano comprising carvacrol, thymol, limonene, pinene, p-cymene and caryophyllene. In certain embodiments, the extract of oregano comprises 50 to 70 weight % carvacrol, 10 to 35 weight % thymol and 10 to 20 weight % p-cymene, based on the weight of the extract of oregano; or 55 to 65 weight % carvacrol, 10 to 35 weight % thymol and 12 to 18 weight % p-cymene, based on the weight of the extract of oregano. In some embodiments, the extract of oregano comprises about 61 weight % carvacrol and 15 weight % p-cymene, based on the weight of the extract of oregano.

In some embodiments, the oral care composition comprises bisabolol and the extract of oregano. In some embodiments, the ratio of the extract of oregano to bisabolol is from 1:1 to 7:1 by weight, from 2:1 to 6:1 by weight, from 3:1 to 5:1 by weight, from 4:1 to 4.5:1 by weight, or about 4.4:1 by weight. In some embodiments, the oral care composition comprises from 150 to 1000 ppm bisabolol and from 500 to 4500 ppm extract of oregano; from 180 to 950 ppm bisabolol and from 800 to 4100 ppm extract of oregano; from 500 to 950 ppm bisabolol and from 2200 to 4200 ppm extract of oregano; or from 850 to 950 ppm bisabolol and from 3900 to 4100 ppm extract of oregano.

In some embodiments, the composition comprises citral and the extract of oregano.

In some embodiments, the active compounds from an extract of rosemary are cyclic monoterpenoid ethers, monoterpenoid alcohols, monoterpenoid ketones, and bicyclic monoterpenes. In some embodiments, the active compounds from an extract of rosemary are α-pinene, β-pinene, borneol, bornyl acetate, camphor, camphene, 1,8-cineole and limonene. Borneo(is a monoterpenoid alcohol, camphor is a monoterpenoid ketone, camphene is a bicyclic monoterpene, and 1,8-cineole (also known as eucalyptol) is a cyclic monoterpenoid ether. In some embodiments, the oral care composition comprises an extract of rosemary, the extract of rosemary comprising α-pinene, β-pinene, borneol, bornyl acetate, camphor, camphene, 1,8-cineole and limonene. In certain embodiments, the extract of rosemary comprises 25 to 40 weight % α-pinene, 5 to 20 weight % β-pinene, and 10 to 25 weight % camphor, based on the weight of the extract of rosemary; or 30 to 40 weight % α-pinene, 10 to 15 weight % β-pinene, and 10 to 15 weight % camphor, based on the weight of the extract of rosemary. In certain embodiments, the extract of rosemary comprises about 36 weight % α-pinene, about 13 weight % β-pinene, and about 16 weight % camphor, based on the weight of the extract of rosemary.

In some embodiments, the oral care composition comprises bisabolol and the extract of rosemary. In some embodiments, the ratio of the extract of rosemary to bisabolol is from 40:1 to 60:1 by weight, from 45:1 to 55:1 by weight, from 46:1 to 50:1 by weight, or about 48:1 by weight. In some embodiments, the oral care composition comprises from 10 to 150 ppm bisabolol and from 800 to 5200 ppm extract of rosemary; from 20 to 110 ppm bisabolol and from 970 to 5000 ppm extract of rosemary; from 50 to 110 ppm bisabolol and from 2800 to 5000 ppm extract of rosemary; or from 90 to 110 ppm bisabolol and from 4800 to 5000 ppm extract of rosemary.

In some embodiments, the composition comprises citral and the extract of rosemary.

In some embodiments, the composition comprises carvacrol and the extract of rosemary. In some embodiments, the ratio of the extract of rosemary to carvacrol is from 60:1 to 90:1 by weight, from 65:1 to 85:1 by weight, from 70:1 to 80:1 by weight, or about 75:1 by weight. In some embodiments, oral care composition comprises from 5 to 80 ppm carvacrol and from 800 to 5300 ppm extract of rosemary; from 10 to 70 ppm carvacrol and from 980 to 5000 ppm extract of rosemary; from 30 to 70 ppm carvacrol and from 2800 to 5000 ppm extract of rosemary; or from 50 to 70 ppm carvacrol and from 4800 to 5000 ppm extract of rosemary.

In some embodiments, the composition comprises thymol and the extract of rosemary.

In some embodiments, the composition comprises the extract of rosemary and an extract of oregano. In sonic embodiments, the extract of oregano comprises carvacrol, thymol, limonene, pinene, p-cymene and caryophyllene. In certain embodiments, the extract of oregano comprises 50 to 70 weight % carvacrol, 10 to 35 weight % thymol and 10 to 20 weight % p-cymene, based on the weight of the extract of oregano.

In certain embodiments, the oral care composition comprises a combination of:

(a) bisabolol and citral,

(b) bisabolol and carvacrol,

(c) carvacrol and thymol,

(d) bisabolol and an extract of oregano,

(e) bisabolol and an extract of rosemary, or

(f) carvacrol and an extract of rosemary;

wherein the ratios and amounts of the bisabolol, citral, carvacrol, thymol, extract of oregano and extract of rosemary in each of combinations (a) to (f) may be as described in any of the above embodiments relating to these combinations.

In some embodiments, the total concentration of the at least one zinc ion source in the oral care composition is from 0.1 to 4.0 weight from 0.3 to 3.0 weight from 0.5 to 2.0 weight %, from 1.0 to 1.9 weight %, from 1.3 to 1.7 weight %, or about 1.5 weight %, based on the total weight of the composition.

In certain embodiments, the at least one zinc ion source is selected from zinc citrate, zinc oxide, zinc acetate, zinc gluconate, zinc glycinate, zinc sulfate, zinc phosphate and sodium zinc citrate. In some embodiments, the composition comprises zinc oxide and zinc citrate. In certain embodiments, the weight ratio of zinc oxide to zinc citrate is from 0:5 to 5:1; from 0:5 to 4:1, or from 0:5 to 3:1. In some embodiments, the weight ratio of zinc oxide to zinc citrate is from 0.1:1 to 4:1, from 1:1 to 3:1, from 1.5:1 to 2.5:1, or about 2:1. In some embodiments, the composition comprises from 0.1 to 5.0 weight % zinc oxide and from 0.1 to 2.0 weight % zinc citrate; from 0.1 to 2.0 weight % zinc oxide and from 0.20 to 1.0 weight' zinc citrate; from 0.5 to 1.5 weight zinc oxide and from 0.25 to 0.75 weight % zinc citrate; from 0.75 to 1.25 weight % zinc oxide and from 0.4 to 0.6 weight % zinc citrate; or about 1.0 weight % zinc oxide and about 0.5 weight % zinc citrate.

In some embodiments, the composition is a dentifrice, a toothpaste, a gel, a tooth a mouthwash, a mouthrinse, a lozenge, a tablet, a spray, a gum, or a film.

In a second aspect, the present invention also provides an oral care composition as described in any of the above embodiments, for use in reducing or inhibiting biofilm formation in an oral cavity.

In a third aspect, the present invention also provides a method of reducing or inhibiting biofilm formation in an oral cavity, the method comprising contacting the oral cavity with an oral care composition as described in any of the above embodiments.

In a fourth aspect, the present invention also provides for the use, in an oral care composition, of a combination of (a) at least one zinc ion source; and (b) at least two active ingredients, wherein said active ingredients are selected from one or more of the following groups: (i) monoterpenoid phenols; (ii) monoterpenoid aldehydes; (iii) sesquiterpenoid alcohols; (iv) active compounds from an extract of oregano; and (v) active compounds from an extract of rosemary; to reduce or inhibit biofilm formation in an oral cavity. The at least one zinc ion source and the at least two active ingredients may be as discussed in any of the above embodiments. Furthermore, the composition may be a dentifrice, a toothpaste, a gel, a tooth powder, a mouthwash, a mouthrinse, a lozenge, a tablet, a spray, a gum, or a film.

In any embodiments of each of the above aspects, the oral care compositions may further comprise additional ingredients. These additional ingredients may include, but are not limited to, diluents, bicarbonate salts, surfactants, foam modulators, sweeteners, flavorants, pigments, antibacterial agents, anticaries agents, anticalculus or tartar control agents, polymers (such as xanthan gum, carboxymethylcellulose, carrageenan gum) and mixtures thereof.

In some embodiments, the oral care compositions of the present invention comprise at least one bicarbonate salt useful for example to impart a “clean feel” to teeth and gums due to effervescence and release of carbon dioxide. The one or more additional bicarbonate salts are optionally present in a total amount of about 0.1 wt. % to about 50 wt. %, for example about 1 wt. % to 20 wt. %, by total weight of the composition.

The oral care compositions of the invention may also comprise at least one surfactant. Any orally acceptable surfactant, most of which are anionic, nonionic or amphoteric, can be used. One or more surfactants are optionally present in a total amount of about 0.01 wt. % to about 10 wt. %, for example, from about 0.05 wt. % to about 5 wt. %, or from about 0.1 wt. % to about 2 wt. % by total weight of the composition.

The oral care compositions of the invention may comprise at least one foam modulator, useful for example to increase amount, thickness or stability of foam generated by the composition upon agitation. One or more foam modulators are optionally present in a total amount of about 0.1 wt. % to about 10 wt. %, for example from about 0.2 wt. % to about 5 wt. %, or from about 0.25 wt. % to about 2 wt. %, by total weight of the composition.

The oral care compositions of the present invention may comprise at least one sweetener (such as, for example, sodium saccharin), useful for example to enhance taste of the composition. One or more sweeteners are optionally present in a total amount depending strongly on the particular sweetener(s) selected, but typically 0.005 wt. % to 5 wt. %, by total weight of the composition, optionally 0.005 wt. % to 0.2 wt. %, further optionally 0.05 wt. % to 0.1 wt. % by total weight of the composition.

The compositions of the present invention may also comprise at least one flavorant, useful for example to enhance taste of the composition. One or more flavorants are optionally present in a total amount of from about 0.01 wt. % to about 5 wt. %, for example, from about 0.03 wt. % to about 2.5 wt. %, optionally about 0.05 wt. % to about 1.5 wt. %, further optionally about 0.1 wt. % to about 0.3 wt. % by total weight of the composition.

The compositions of the invention may comprise at least one colorant. Colorants herein include pigments, dyes, lakes and agents imparting a particular luster or reflectivity such as pearling agents. Any orally acceptable colorant can be used. One or more colorants are optionally present in a total amount of from about 0.001 wt. % to about 20 wt. %, for example, from about 0.01 wt. % to about 10 wt. %, or from about 0.1 wt. % to about 5 wt. %, by total weight of the composition.

The oral care compositions may also comprise a fluoride ion source. Fluoride ion sources may be added to the compositions of the invention at a level of about 0.001 wt. % to about 10 wt. %, e.g., from about 0.003 wt. % to about 5 wt. %, 0.01 wt. % to about 1 wt., or about 0.05 wt. %. However, it is to be understood that the weights of fluoride salts to provide the appropriate level of fluoride ion will obviously vary based on the weight of the counter ion in the salt, and one of skill in the art may readily determine such amounts.

The compositions of the present invention may comprise a saliva stimulating agent useful, for example, in amelioration of dry mouth. One or more saliva stimulating agents are optionally present in saliva stimulating effective total amount.

The compositions of the present invention may include antisensitivity agents. Such agents may be added in effective amounts, e.g., from about 1 wt. % to about 20 wt. % by weight based on the total weight of the composition, depending on the agent chosen.

The composition of the invention may further comprise an antioxidant.

The compositions of the present invention may additionally optionally comprise a tartar control (anticalculus) agent.

### EXAMPLES

### Example 1

Compositions 1 to 9 were formulated, each of which contained 1 weight % zinc oxide, 0.5 weight % zinc citrate and two active ingredients selected from bisabolol, citral, carvacrol and thymol. For each combination of the aforementioned active ingredients, three dentifrices were formulated which differed only in the total concentration of the two active ingredients (the total concentration being 1000 ppm, 3000 ppm and 5000 ppm). The weight ratio of the two active ingredients for each particular comibination was kept constant. The base dentifrice formulation was the same for each of compositions 1 to 9, with only the balance of water in the composition being adjusted to allow for the differences in concentration of the two active ingredients.

The dentifrice compositions 1 to 9 are shown in Table 1, below:

TABLE 1

| | Bisabolol | Citral | Carvacrol | Thymol |
|---|---|---|---|---|
| Formula | (ppm) | (ppm) | (ppm) | (ppm) |
| 1 | 335.1 | 664.9 | | |
| 2 | 1005.3 | 1994.7 | | |
| 3 | 1675.5 | 3324.5 | | |
| 4 | 609.8 | | 390.2 | |
| 5 | 1829.4 | | 1170.6 | |
| 6 | 3049.0 | | 1951.0 | |
| 7 | | | 81.6 | 918.4 |
| 8 | | | 244.8 | 2755.2 |
| 9 | | | 408.0 | 4592.0 |

The Biofilm Growth Inhibition University of Manchester Model was used to determine the ability of the above dentifrices 1 to 9 to reduce oral biofilms. The ability of a Control A dentifrice (which had the same base formulation as compositions 1 to 9 and contained 1 weight % zinc oxide and 0.5 weight % zinc citrate, but contained no bisabolol, citral, carvacrol or thymol) and a placebo dentifrice (which had the same base formulation as compositions 1 to 9, but contained no zinc ion sources and no bisabolol, citral, carvacrol or thymol) to reduce oral biofilms was also tested.

The protocol for this model is as follows
(1) Dental plaque was collected from four healthy volunteers and pooled together as inoculum. The Optical Density of the inoculum was matched to 0.3 absorbance at 610 nm.
(2) Sterile hydroxyapatite (HAP) disks were incubated under anaerobic conditions at 37° C. for 24 hours with 1 mL of sterile artificial saliva (with 0.01 weight % sucrose) and 1 mL of pooled saliva in a 24 well microplate.
(3) For each test dentifrice (and for each control) a treatment solution of 1 part dentifrice: 2 parts sterile distilled water by weight was made up. Each freshly prepared treatment solution was added to three wells and allowed to contact the HAP disk therein for 10 minutes.
(4) The liquid phase of each well was then removed and was replaced by 2 mL sterile artificial saliva.
(5) The disks were then maintained at 37° C. under anaerobic conditions for 8 days.
(6) At intervals of 2, 4 and 8 days, the disks were collected aseptically and transferred to half-strength pre-reduced thioglycollate medium (4.5 mL per disk).
(7) 100 μL of the dilution 10-4, 10-5 and 10-6 were plated in duplicates for each disk on Neomycin/Vancomycin (NV) Agar for Total Gram-negative Anaerobes.
(8) The plates were surface-spread using a sterile spreader and were incubated anaerobically at 37° C. for 72 hours, after which time the number of colonies on each plate was counted.

The log10 CFU/ml (where CFU=colony forming units) for each composition was calculated. A lower Log10 CFU/ml indicates that the composition tested has greater efficacy in inhibiting biofilm growth.

The results of the tests are shown in Table 2, below:

TABLE 2

| Formula | Avg log CFU/mL |
|---|---|
| 1 | 6.43 |
| 2 | 6.36 |
| 3 | 6.09 |
| 4 | 6.50 |
| 5 | 6.38 |
| 6 | 6.17 |
| 7 | 6.44 |
| 8 | 6.42 |
| 9 | 6.27 |
| Control A | 6.70 |
| Placebo | 8.11 |

As can be seen from Table 2, all of the compositions 1 to 9 provided greater efficacy in reducing biofilm than the Control A formulation (which contained zinc oxide and zinc citrate, but no bisabolol, citral, carvacrol or thymol) and the Placebo.

### Example 2

Compositions 10 to 18 were formulated, each of which contained 1 weight % zinc oxide, 0.5 weight % zinc citrate and two active ingredients selected from bisabolol, carvacrol, oregano essential oil (an extract of oregano) and rosemary essential oil (an extract of rosemary). For each combination of the aforementioned active ingredients, three dentifrices were formulated which differed only in the total concentration of the two active ingredients (the total concentration being 1000 ppm, 3000 ppm and 5000 ppm). The weight ratio of the two active ingredients for each particular combination was kept constant. The base dentifrice formulation was the same for each of compositions 10 to 18, with only the balance of water in the composition being adjusted to allow for the differences in concentration of the two active ingredients.

The dentifrice compositions 10 to 18 are shown in Table 3, below:

TABLE 3

| | Bisabolol | Carvacrol | Oregano | Rosemary |
|---|---|---|---|---|
| Formula | (ppm) | (ppm) | (ppm) | (ppm) |
| 10 | 185.2 | | 814.8 | |
| 11 | 555.6 | | 2444.4 | |
| 12 | 926.0 | | 4074.0 | |
| 13 | 20.4 | | | 979.6 |
| 14 | 61.2 | | | 2938.8 |
| 15 | 102.0 | | | 4898.0 |
| 16 | | 13.2 | | 986.8 |
| 17 | | 39.6 | | 2960.4 |
| 18 | | 66.0 | | 4934.0 |

The Biofilm Growth Inhibition University of Manchester Model was used to determine the ability of the above dentifrices 1 to 9 to reduce oral biofilms. The Control data for this test was not available.

The protocol for this model is as follows
(9) Dental plaque was collected from four healthy volunteers and pooled together as inoculum. The Optical Density of the inoculum was matched to 0.3 absorbance at 610 nm.
(10) Sterile hydroxyapatite (HAP) disks were incubated under anaerobic conditions at 37° C. for 24 hours with 1 mL of sterile artificial saliva (with 0.01 weight % sucrose) and 1 mL of pooled saliva in a 24 well microplate.
(11) For each test dentifrice (and for each control) a treatment solution of 1 part dentifrice: 2 parts sterile distilled water by weight was made up. Each freshly prepared treatment solution was added to three wells and allowed to contact the HAP disk therein for 10 minutes.
(12) The liquid phase of each well was then removed and was replaced by 2 mL sterile artificial saliva.
(13) The disks were then maintained at 37° C. under anaerobic conditions for 8 days.
(14) At intervals of 2, 4 and 8 days, the disks were collected aseptically and transferred to half-strength pre-reduced thioglycollate medium (4.5 mL per disk).
(15) 1000 μL of the dilution 10-4, 10-5 and 10-6 were plated in duplicates for each disk on Neomycin/Vancomycin (NV) Agar for Total Gram-negative Anaerobes.
(16) The plates were surface-spread using a sterile spreader and were incubated anaerobically at 37° C. for 72 hours, after which time the number of colonies on each plate was counted.

The log10 CFU/ml (where CFU=colony forming units) for each composition was calculated. A lower Log10 CFU/ml indicates that the composition tested has greater efficacy in inhibiting biofilm growth.

The results of the tests are shown in Table 4. below:

TABLE 4

| Formula | Avg log CFU/mL |
|---|---|
| 10 | 4.98 |
| 11 | 4.72 |
| 12 | 4.54 |
| 13 | 4.94 |
| 14 | 4.65 |
| 15 | 4.46 |
| 16 | 4.86 |
| 17 | 4.52 |
| 18 | 4.38 |
| Control | Not Available |

As can be seen from Table 4, all of the compositions 1 to 9 provided excellent efficacy in reducing biofilm. The control data for this test was not available, but it is believed that the same improvement in efficacy would be achieved with compositions 10-18 as was achieved with compositions 1 to 9.

### Example 3

A commercially available zinc citrate/stannous chloride dentifrice that contained no bisabolol, citral, carvacrol, thymol, oregano essential oil or rosemary essential oil (Comparative Commercial Product) was tested for comparison.

TABLE 5

| | |
|---|---|
| Comparative Commercial | 6.49 |
| Product | |

Compositions 1-18 provided great efficacy in reducing biofilm over the commercially available zinc citrate/stannous chloride dentifrice which did not contain bisabolol, citral, carvacrol, thymol, oregano essential oil or rosemary essential oil.

What is claimed is:

1. An oral care composition comprising:
(a) at least one zinc ion source; and
(b) at least two active ingredients, wherein said active ingredients are selected from one or more of the following groups:
(i) a monoterpenoid phenol,
(ii) a monoterpenoid aldehyde,
(iii) a sesquiterpenoid alcohol,
(iv) an active compound from an extract of oregano, and
(v) an active compound from an extract of rosemary.

2. The oral care composition of claim 1, wherein the total concentration of the at least one zinc ion source is from 0.1 to 4.0 weight %, based on the total weight of the composition.

3-5. (canceled)

6. The oral care composition of claim 1, wherein the composition comprises at least one monoterpenoid phenol selected from carvacrol and thymol.

7. The oral care composition of claim 6, wherein the composition comprises carvacrol.

8. The oral care composition of claim 6, wherein the composition comprises thymol.

9. The oral care composition of claim 1, wherein the composition comprises a monoterpenoid aldehyde, and wherein the monoterpenoid is citral.

10. The oral care composition of claim 1, wherein the composition comprises a sesquiterpenoid alcohol, and wherein the sesquiterpenoid alcohol is bisabolol.

11. The oral care composition of claim 1, wherein the composition comprises bisabolol and citral.

12. The oral care composition of claim 11, wherein the ratio of citral to bisabolol is from 1:1 to 3:1 by weight.

13. The oral care composition of claim 1, wherein the composition comprises bisabolol and carvacrol.

14. The oral care composition of claim 13, wherein the ratio of bisabolol to carvacrol is from 1:2 to 2:1 by weight.

15-17. (canceled)

18. The oral care composition of claim 1, wherein the composition comprises carvacrol and thymol.

19. The oral care composition of claim 18, wherein the ratio of thymol to carvacrol is from 8:1 to 13:1 by weight.

20. The oral care composition of claim 1, wherein the oral care composition comprises an active compound from an extract of oregano, and wherein the active compound from an extract of oregano is selected from carvacrol, thymol, limonene, pinene, p-cymene, caryophyllene, and mixtures thereof.

21. The oral care composition of claim 20, wherein the active compound from the extract of oregano comprises 50 to 70 weight % carvacrol, 10 to 35 weight % thyrnol and 10 to 20 weight % p-cymene, based on the total weight of the extract of oregano.

22. The oral care composition of claim 20, wherein the composition further comprises bisabolol.

23. The oral care composition of claim 22, wherein the ratio of the active compound from an extract of oregano to bisabolol is from 2:1 to 6:1 by weight.

24. (canceled)

25. The oral care composition of claim 1, wherein the oral care composition comprises an active compound from an extract of rosemary, and wherein the active compound from the extract of rosemary is selected from α-pinene, β-pinene, borneol, bornyl acetate, camphor, camphene, 1,8-cineole, limonene, and mixtures thereof.

26. The oral care composition of claim 25, wherein the extract of rosemary comprises 25 to 40 weight % α-pinene, 5 to 20 weight % β-pinene, and 10 to 25 weight % camphor, based on the total weight of the extract of rosemary.

27. The oral care composition of claim 25, wherein the composition further comprises bisabolol.

28. The oral care composition of claim 27, wherein the ratio of the active compound from an extract of rosemary to bisabolol is from 40:1 to 60:1 by weight.

29. (canceled)

30. The oral care composition of claim 25, wherein the composition further comprises carvacrol.

31. The oral care composition of claim 30, wherein the ratio of the active compound from an extract of rosemary to carvacrol is from 70:1 to 80:1 by weight.

32. (canceled)

33. The oral care composition of claim 25, wherein the composition further comprises an active compound from an extract of oregano.

34. The oral care composition of claim 33, wherein the active compound from an extract of oregano is selected from carvacrol, thymol, limonene, pinene, p-cymene, caryophyllene, and mixtures thereof.

35. The oral care composition of claim 33, wherein the extract of oregano comprises 50 to 70 weight % carvacrol, 10 to 35 weight % thymol and 10 to 20 weight % p-cymene, based on the weight of the extract of oregano.

36. The oral care composition of claim 1, wherein the at least one zinc ion source is selected from zinc citrate, zinc oxide, zinc acetate, zinc gluconate, zinc glycinate, zinc sulfate, zinc phosphate and sodium zinc citrate.

37. The oral care composition of claim 36, wherein the composition comprises zinc oxide and zinc citrate.

38. The oral care composition of claim 37, wherein the weight ratio of zinc oxide to zinc citrate is from 0:5 to 3:1.

39. (canceled)

40. The oral care composition of claim 37, wherein the composition comprises from 0.1 to 2.0 weight % zinc oxide and from 0.20 to 1.0 weight % zinc citrate.

41. The oral care composition of claim 1, wherein the composition is a dentifrice, a toothpaste, a gel, a tooth powder, a mouthwash, a mouthrinse, a lozenge, a tablet, a spray, a gum, or a film.

42. The oral care composition of claim 1, for use in reducing or inhibiting biofilm formation in an oral cavity.

43. A method of reducing or inhibiting biofilm formation in an oral cavity, the method comprising contacting the oral cavity with an oral care composition according to claim 1.

44. (canceled)
